(19) United States
(12) Patent Application Publication
(10) Pub. No.: US 2004/0181166 A1
(43) Pub. Date: Sep. 16, 2004

(54) Body temperature sensing and indicating and teeth protection system

(76) Inventors: David S. Williford, La Porte, TX (US); Bruce D. Butler, Houston, TX (US); Steven J. Ritter, Houston, TX (US); Phyllis Butler, Houston, TX (US); Wayne Price, San Antonio, TX (US)

Correspondence Address: DUANE, MORRIS, LLP, SUITE 3150, 3200 SOUTHWEST FREEWAY, HOUSTON, TX, 77046, US

(21) Appl. No.: 10/388,356

(22) Filed: Mar. 13, 2003

(51) Int. Cl. A61B 5/00; A61C 5/14

(52) U.S. Cl. 600/549; 128/861

(57) ABSTRACT

This invention is directed to a body temperature sensing and indicating teeth protection system. The present invention is capable of sensing and indicating a user's body temperature while also protecting one or more teeth of the user.

### BACKGROUND OF THE INVENTION

1. Field of the Invention

This invention is directed to a body temperature sensing and indicating and teeth protection system. The present invention is capable of sensing and indicating a user's body temperature while also protecting one or more teeth of the user.

2. Description of the Prior Art

Children and adults engaging in physical activity, including athletics or physical training, can suffer from heat exhaustion or heat stroke. The consequences of heat stroke can be fatal. In many sports, it is desirable to wear a mouth guard or other protective head gear in order to protect against injury to the teeth and/or head of the participant. There is utility in providing a body temperature sensing and indicating device combined with a teeth casing which can be worn by participants in athletic activities or physical training where heat stroke or heat exhaustion is a potential danger.

Body temperature sensing and indicating devices exist in the prior art. Such devices include mercury filled thermometers, and electronic thermometers comprising digital temperature displays. Prior art temperature sensing and/or indicating devices are disclosed in U.S. Pat. Nos. 5,559,497; 5,534,013; 4,447,164; 3,980,581; 5,033,864; and 5,021,060. Prior art mouth guards also exist. Such mouth guards are disclosed in U.S. Pat. Nos. 4,502,794; 4,791,941; 5,293,880; and 6,092,524.

The prior art does not disclose a system containing a teeth casing and a temperature sensing and indicating device which maybe worn by children or adults engaged in physical activity where heat stroke or heat exhaustion is a potential danger.

### SUMMARY OF THE INVENTION

The present invention is directed toward a system that fulfills the functions of encasing one or more teeth, sensing body temperature, and indicating body temperature. The present invention comprises a temperature sensor, a temperature indicator, and a teeth casing.

### DESCRIPTION OF THE FIGURES

FIG. 1a is a top view of a first embodiment of the present invention.

FIG. 1b is a side view of a second embodiment of the present invention.

FIG. 1c is an isometric view of a first embodiment of the present invention.

FIG. 2a is a block diagram of an electronic embodiment of the present invention.

FIG. 2b is a block diagram of another embodiment of the present invention.

FIG. 3a is an isometric view of a third embodiment of the present invention.

FIG. 3b is a block diagram of a multiple senor embodiment of the present invention.

FIG. 4 is an isometric view of a fourth embodiment of the present invention.

### DESCRIPTION OF THE PREFERRED EMBODIMENTS

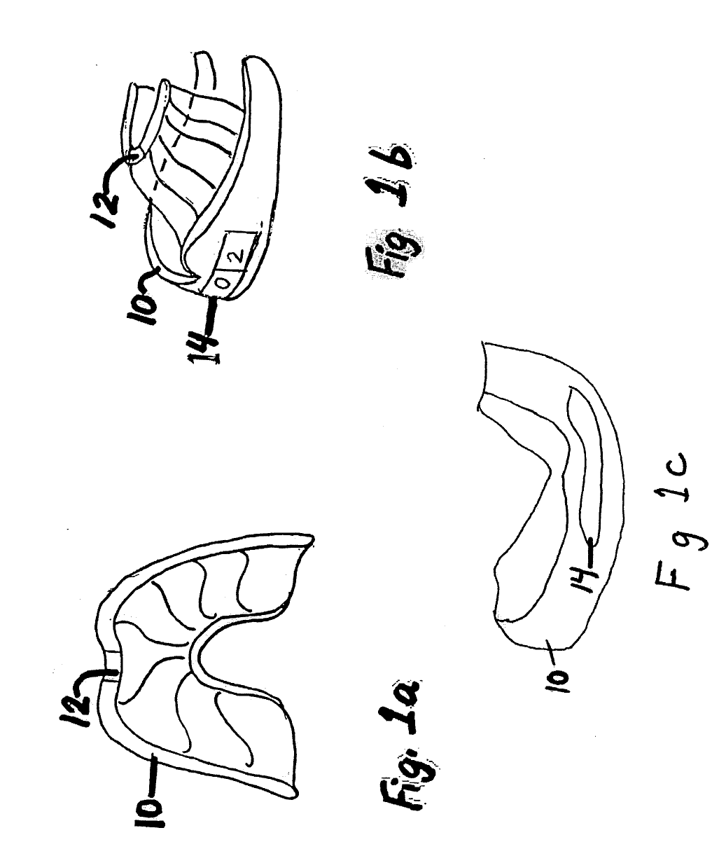

The present invention is directed toward a body temperature sensing and indicating and teeth protection system comprising a teeth casing 10 sized and adapted to contain at least one tooth located in a wearer's mouth. In one preferred embodiment, the casing is an athletic activity mouth guard as shown in FIGS. 1a-1c. In another embodiment, the teeth casing is shaped to extend in front of, and behind, any teeth which it contains as shown in FIGS. 1a-1b. In another preferred embodiment, the teeth casing is sized and shaped to form a channel adapted to contain at least one tooth located in a wearer's mouth, as shown in FIGS. 1a-1c.

In a preferred embodiment, the casing is flavored with a flavor that is appealing to wearers. Flavoring may be achieved by soaking the casing in a flavored solution for a sufficient amount of time for the casing to absorb the flavoring. In one preferred embodiment, the flavored solution contained mint extract and results in a mint flavored casing after the soaking process described above.

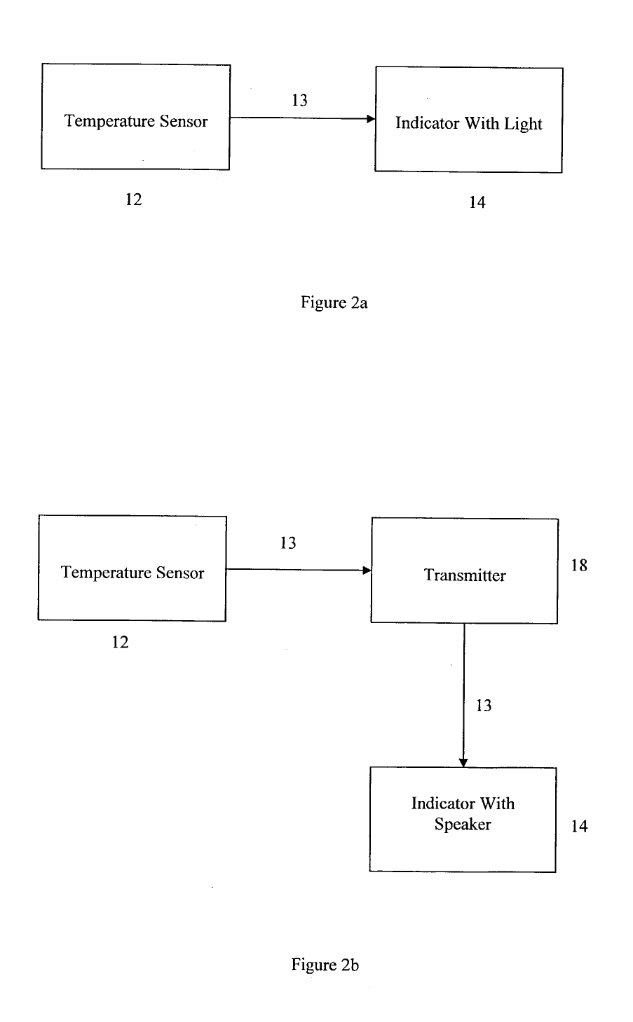

The invention further comprises a temperature sensor 12 connected to the casing and positioned such that when the casing is inserted in a wearer's mouth, the sensor senses the wearer's body temperature and produces a signal 13 indicating the wearer's body temperature as shown in FIGS. 1a-1b and 2a. In a preferred embodiment, the temperature sensor is positioned to contact the gingiva or contact the adjacent tissue between the gingiva and the inner surface of the wearer's lip, when the casing is inserted in the wearer's mouth, as shown in FIG. 1a.

In another preferred embodiment, the temperature sensor is positioned to contact the roof of the wearer's mouth when the casing is inserted in the wearer's mouth, as shown in FIG. 1b. In this embodiment, the sensor may contact the palate. In another preferred embodiment, the sensor is positioned to contact both the wearer's gingiva and the wearer's palate.

The invention further comprises a temperature indicator 14 operatively connected to the sensor to receive the signal as shown in FIG. 2a. The temperature indicator is positioned to indicate the wearer's body temperature as shown in FIG. 1b. In another embodiment, the invention further comprises a temperature transmitter 18 coupled to the sensor and configured to transmit a signal 13 indicating a wearer's body temperature as shown in FIG. 2b. In this embodiment, the invention further comprises a temperature indicator remotely located from the temperature sensor and the temperature transmitter. The depiction of signal 13 in FIG. 2b using a vertical line is not intended to express or imply that this is a hard wired connection between the transmitter and the indicator. The transmission of the signal from the transmitter to the indicator may be by any wireless means of data transfer known to those skilled in the art. The vertical line connecting the transmitter and the indicator in FIG. 2b is intended to depict the transfer of data from the transmitter to the indicator via signal 13. The indicator is configured to receive the signal from the transmitter and to indicate a wearer's body temperature in response to the signal as shown in FIG. 2b.

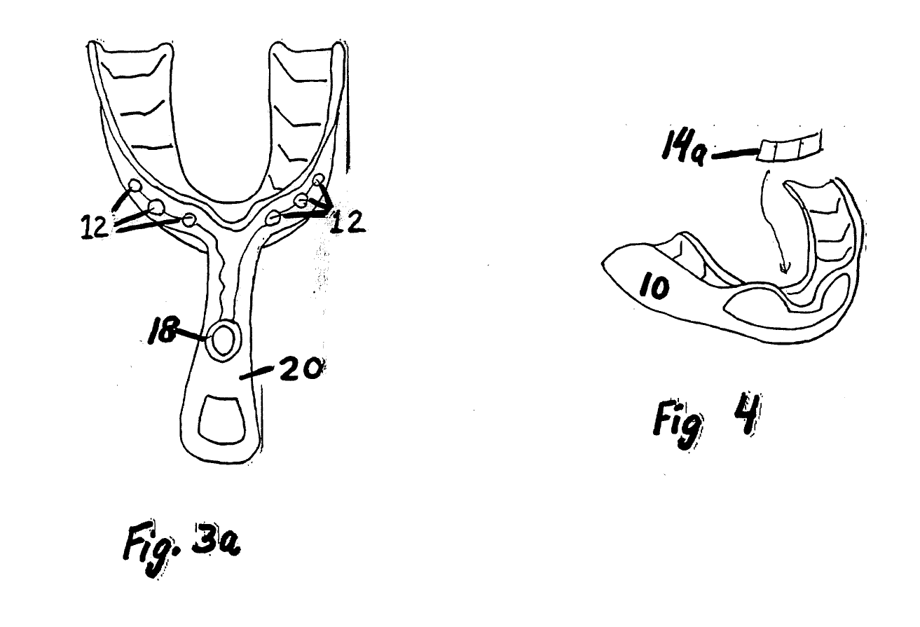

In one preferred embodiment, the casing comprises a head gear connector 20 as shown in FIG. 3a. In this preferred embodiment, the transmitter 18 may be mounted on the head gear connector, as shown in FIG. 3a.

In one preferred embodiment, as shown in FIG. 1c, the temperature sensor 14 is adapted to employ a chemical reaction to sense body temperature. In this embodiment, the temperature indicator is colorimetric. FIG. 1c depicts an embodiment of a colorimetric indicator 14 which changes color when a preselected temperature is measured by the temperature sensor. Such colorimetric sensors are available from Hallcrest Products, Inc. of Glenview, Ill.

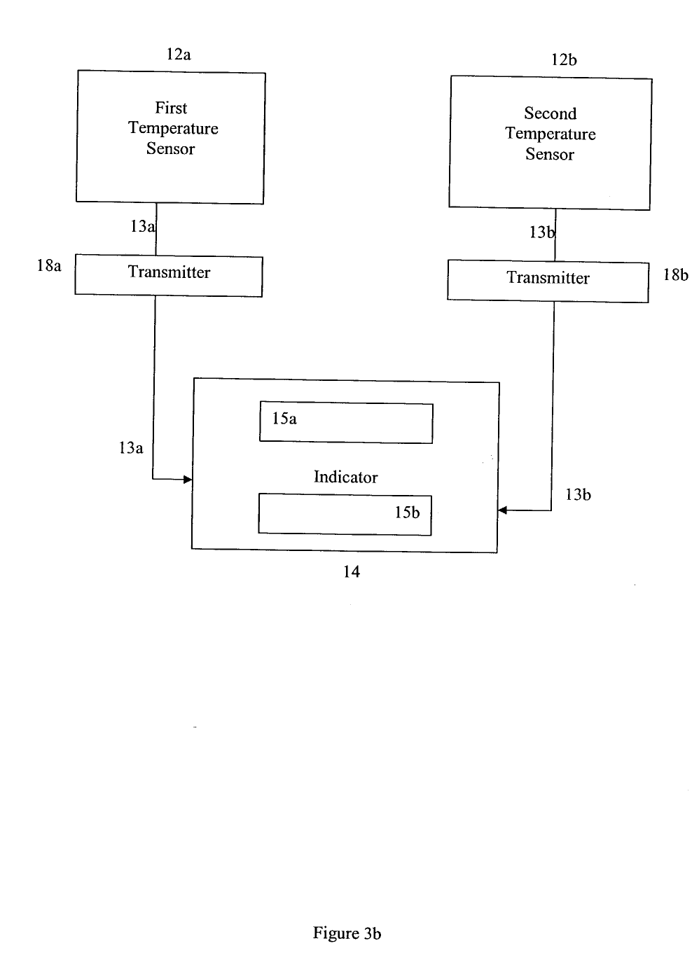

In another preferred embodiment, the temperature indicator comprises a removable insert 14a, as shown in FIG. 4. In another preferred embodiment, the colorimetric indicator is adapted to display a sequence of at least two colors in response to increasing temperature sensed by the sensor up to a maximum temperature in the range of 98-110° F. In a preferred embodiment, the color sequence is reversible and responds to deceasing temperature sensed by the sensor. Multicolor colorimetric temperature indicators are available from Hallcrest Products, Inc. of Glenview, Ill. Reversible temperature indicators are available from Lab Safety Supply, Inc. of Janesville, Wis. In another preferred embodiment where the indicator is adapted to display a sequence of at least two colors in response to increasing temperature, the color sequence is irreversible. Irreversible temperature indicators, suitable for use in the present invention are available from Omega Engineering, Inc. of Stamford, Conn., and from Lab Safety Supply, Inc. of Janesville, Wis. In another preferred embodiment, the indicator is perceivable to a person positioned anterior to the teeth casing, as shown in FIGS. 1a-1c, and 4.

One embodiment of the present invention comprises an electronic temperature sensor and an electronic temperature indicator, as shown in FIG. 2a. An electronic temperature sensor and an electronic temperature indicator suitable for use with the present invention are available from Safety 1st, Inc. of Canton, Mass. In this embodiment, an electronic temperature sensor is connected to the casing and positioned such that when the casing is inserted in a wearer's mouth, the electronics sensor senses the wearer's body temperature and produces a signal indicative of the wearer's body temperature.

This embodiment further comprises an electronic temperature indicator, operatively connected to the sensor to receive the signal and position to indicate the wearer's body temperature. In a preferred embodiment, the indicator comprises at least 2 digits, as shown in FIG. 1b. In another preferred embodiment, the indicator comprises a light as shown in FIG. 2a. In yet another preferred embodiment, the indicator is configured to produce an audible signal in response to the temperature sensor, as shown in FIG. 2b. In another preferred embodiment, the indicator is digital and it is positioned to be located anterior to the teeth of a wearer of the casing, as shown in FIG. 1b.

The present invention may also be used to protect the teeth and sense the temperature of multiple wearers. A multiple wearer embodiment of the present invention is directed toward use by at least two wearers. In this embodiment, the invention comprises a first teeth casing sized and shaped to contain at least one tooth located in a first wearer's mouth and a second teeth casing sized and shaped to contain at least one tooth located in a second wearer's mouth. The first and second teeth casings may be of the type shown in FIGS. 1a-1c.

This embodiment further comprises a first temperature sensor 12a connected to the first casing and positioned such that when the first casing is inserted in a first wearer's mouth the first sensor senses the first wearer's body temperature and produces the first signal 13a indicating the first wearer's body temperature as shown in FIG. 3b. This embodiment further comprises a second temperature sensor 12b connected to the second casing and positioned such that when the second casing is inserted in a second wearer's mouth, the sensor senses the second wearer's body temperature and produces the second signal 13b indicating the second wearer's body temperature as shown in FIG. 3b. First and second transmitters 18a and 18b, respectively, are configured to receive signals 13a and 13b, respectively.

This embodiment further comprises a temperature indicator 14 operatively connected to the first and second transitters to receive said first and second signals, as shown in FIG. 3b. The indicator is positioned remotely from the sensors to indicate the body temperature of each wearer, as shown in FIG. 3b. In a preferred embodiment, the indicator comprises a first display 15a operatively coupled to the first sensor to display the body temperature of a first wearer and a second display 15b operatively coupled to the second sensor to display the body temperature of a second wearer, as shown in FIG. 3b. This operative coupling may be by wireless data transmission means known to those skilled in the art.

The general configuration shown in FIG. 3b may be adapted for use with more than 2 users. In the case of N users, where N>2, there would be N teeth casings, N temperature sensors, and N temperature signals. In a preferred embodiment, the temperature indicator could also comprise N displays. In an alternate embodiment, the indicator could contain a switching mechanism which allows a single display to selectively display a temperature resulting from any one of the N signals.

The foregoing disclosure and description of the invention are illustrative and explanatory. Various changes in the size, shape, and materials, as well as in the details of the illustrative construction may be made without departing from the spirit of the invention.

What is claimed is:

1. A body temperature sensing and indicating and teeth protection system comprising:
a. a teeth casing sized and shaped to contain at least one tooth located in a wearer's mouth;
b. a temperature sensor connected to the casing and positioned such that when the casing is inserted in a wearer's mouth, the sensor senses the wearer's body temperature and produces a signal indicating the wearer's body temperature; and
c. a temperature indicator operatively connected to the sensor to receive said signal and positioned to indicate the wearer's body temperature.

2. The system of claim 1 wherein the casing is an athletic activity mouth guard.

3. The system of claim 1 wherein the temperature sensor is positioned to contact the gingiva when the casing is inserted in the wearer's mouth.

4. The system of claim 3 wherein the temperature sensor is positioned to contact the wearer's palate.

5. The system of claim 1 wherein the temperature sensor is positioned to contact the adjacent tissue between the gingiva and the inner surface of the wearer's lip.

6. The system of claim 1 wherein the temperature sensor is positioned to contact the roof of the wearer's mouth when the casing is inserted in the wearer's mouth.

7. The system of claim 1 wherein:
a. the temperature sensor is adapted to employ a chemical reaction to sense body temperature; and
b. the temperature indicator is colorimetric.

8. The system of claim 7, wherein the temperature indicator comprises a removable insert.

9. The system of claim 7, wherein the colorimetric indicator is adapted to display a sequence of at least two colors in response to increasing temperatures sensed by the sensor up to a maximum temperature in the range of 98-110° F.

10. The system of claim 9, wherein the color sequence is reversible in response to decreasing temperature sensed by the sensor.

11. The system of claim 9, wherein the color sequence is irreversible.

12. The system of claim 1, wherein the indicator is perceivable to a person positioned anterior to the teeth casing.

13. A body temperature sensing and indicating and teeth protection system comprising:
a. a teeth casing sized and shaped to contain at least one tooth located in a wearer's mouth and shaped to extend in front of and behind any teeth contained therein;
b. an electronic temperature sensor connected to the casing and positioned such that when the casing is inserted in a wearer's mouth, the electronic sensor senses the wearer's body temperature and produces a signal indicative of the wearer's body temperature; and
c. an electronic temperature indicator operatively connected to the sensor to receive said signal and positioned to indicate the wearer's body temperature.

14. The system of claim 13, wherein the indicator comprises at least two digits.

15. The system of claim 13, wherein the indicator comprises a light.

16. The system of claim 13, wherein the indicator is configured to produce an audible signal in response to the temperature sensor.

17. The system of claim 13, wherein the indicator is digital and is positioned to be located anterior to the teeth of a wearer of the casing.

18. A body temperature sensing and indicating and teeth protection system comprising:
a. a teeth casing sized and shaped to form a channel adapted to contain at least one tooth located in a wearer's mouth;
b. a temperature sensor connected to the casing and positioned such that when the casing is inserted in the wearer's mouth, the sensor senses the wearer's body temperature;
c. a temperature transmitter coupled to said temperature sensor and configured to transmit a signal indicating a wearer's body temperature; and
d. a temperature indicator remotely located from the temperature sensor and the temperature transmitter, said indicator being configured to receive said signal from the transmitter and to indicate the wearer's body temperature in response to said signal.

19. The system of claim 18, wherein the casing comprises a head gear connector.

20. The system of claim 18, wherein the transmitter is mounted on the head gear connector.

21. The system of claim 18 wherein the casing is flavored.

22. A body temperature sensing and indicating and teeth protection system comprising:
a. a first teeth casing sized and shaped to contain at least one tooth located in a first wearer's mouth;
b. a first temperature sensor operatively connected to the first casing and positioned such that when the first casing is inserted in a first wearer's mouth, the first sensor senses the first wearer's body temperature and produces a first signal indicating the first wearer's body temperature;
c. a second teeth casing sized and shaped to contain at least one tooth located in a second wearer's mouth;
d. a second temperature sensor operatively connected to the second casing and positioned such that when the second casing is inserted in a second wearer's mouth, the sensor senses the second wearer's body temperature and produces a second signal indicating the second wearer's body temperature;
e. a first transmitter operatively connected to the first sensor to receive the first signal;
f. a second transmitter operatively connected to the second sensor to receive the second signal; and
g. a temperature indicator operatively connected to the first and second transmitters to receive said first and second signals and positioned remotely from the sensors to indicate the body temperature of each wearer.

23. The system of claim 22 wherein the temperature indicator comprises:
a. a first display coupled to receive the first signal from the first sensor and to display the body temperature of a first wearer; and
b. a second display coupled to receive the second signal from the second sensor and to display the body temperature of a second wearer.
